(19) United States
(12) Patent Application Publication
(10) Pub. No.: US 2010/0267984 A1
(43) Pub. Date: Oct. 21, 2010

(54) OXYFLUOROPHOSPHATE SYNTHESIS PROCESS AND COMPOUND THEREFROM

(75) Inventors: Shengshui Zhang, Olney, MD (US); Conrad Xu, North Potamac, MD (US); T. Richard Jow, Potomac, MD (US)

Correspondence Address: U S ARMY RESEARCH LABORATORY;ATTN: RDRL-LOC-I, 2800 POWDER MILL RD, ADELPHI, MD, 20783-1197, US

(73) Assignee: U.S. Government as represented by the Secretary of the Army, Adelphi, MD (US)

(21) Appl. No.: 12/826,843

(22) Filed: Jun. 30, 2010

Related U.S. Application Data
(62) Division of application No. 11/518,743, filed on Sep. 7, 2006.

(51) Int. Cl. C07C 69/67; C07C 69/63

(52) U.S. Cl. 560/187; 560/195

(57) ABSTRACT

An electrolyte compound has the formula

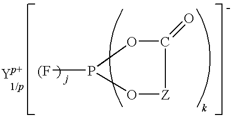

where p is an integer from 1 to 3 inclusive; and Y^{p+} is a metal ion, onium species, or proton; j is an integer value between 0 and 4 inclusive; k is an integer between 1 and 3 inclusive; and the sum 2k and j equals 6; Z is independently in each occurrence CR¹R² or C(O); R¹ and R² are independently in each occurrence H, F or CH₃. A process for preparing an oxyfluorophosphate is also provided.

### RELATED APPLICATIONS

This application is a division of U.S. patent application Ser. No. 11/518,743 filed 7 Sep. 2006 now

### GOVERNMENT INTEREST

The invention described herein may be manufactured, used, and licensed by or for the United States Government.

### FIELD OF THE INVENTION

The present invention generally relates to a process for the synthesis of an oxyfluorophosphate and in particular to a process for preparing an oxyfluorophosphate through the reaction of a phosphorus pentafluoride complex and a metal alkoxy or an ester.

### BACKGROUND OF THE INVENTION

Rechargeable lithium ion batteries have been commercially available for well over a decade. Lithium hexafluorophosphate is commonly employed as the electrolyte salt in lithium ion batteries. Lithium hexafluorophosphate (LiPF₆) is characterized by solubility in aprotic solvents that results in an electrolyte characterized by high electrical conductivities and electrochemical stability. However, lithium hexafluorophosphate has limited applicability in future lithium ion batteries owing to a lack of thermal stability. In solution, lithium hexafluorophosphate dissociates into lithium fluoride and phosphorus pentafluoride which are then free to cationically polymerize electrolyte solvents. Additionally, lithium hexafluorophosphate releases hydrofluoric acid upon contact with moisture. Lithium hexafluorophosphate hydrolysis not only impedes safe handling but also leads to the degradation of transition metal oxides often utilized in electrochemical cells as a cathode material.

Considerable efforts have been made to develop alternative conducting salts to lithium hexafluorophosphate. Representative of these efforts is U.S. Pat. No. 4,505,997 that describes the use of lithium bis(trifluoromethylsulfonyl) imide and lithium tris(trifluoromethylsulfonyl)methanide salts for use in battery electrolytes. U.S. Pat. Nos. 5,874,616 and 6,319,428 describe the use of lithium perfluoro amide salts as battery electrolytes. While these salts display high anodic stability and form solutions having high electrical conductivity with organic carbonates, these same salts suffer the limitation of not adequately passivating aluminum. This is problematic since aluminum is a commonly used current collector for battery cathodes. Additionally, these salts tend to be comparatively difficult to produce and purify.

U.S. Pat. Nos. 6,210,830 and 6,423,454 describe perfluoro- or partially fluorinated-alkyl fluorophosphates as lithium ion battery electrolytes. While the thermal stability and hydrolysis resistance of these compounds as lithium salts are superior to lithium hexafluorophosphate, these salts are comparatively difficult to produce and as such significantly add to production costs for lithium ion batteries containing these salts. Barthel et al. (Journal of Electrochemical Society, 147, 2000, 21) teaches lithium organoborates as an electrolyte salt. These salts have met with limited acceptance owing to the inability to withstand high anodic potentials and the formation of unstable triorganoboranes.

DE 19829030 C1 and U.S. Pat. No. 6,506,516 describe lithium bisoxalatoborate as a battery electrolyte salt. Xu et al. (Electrochemical and Solid-State Letters, 5, 2002, A26) note that lithium bisoxalatoborates readily passivate aluminum, show good thermal stability, yet have met with limited acceptance owing to the poor solubility of bisoxalatoborate in conventional lithium ion battery organic solvents.

U.S. Pat. Nos. 6,407,232; 6,461,773; and 6,485,868 teach a class of cyclic compounds, some of which are lithium salts that appear to offer lithium ion battery salts having good overall properties. However, the process of synthesizing such salts is inherently dangerous and inefficient. Thus, there exists a need for an efficient process for the production of lithium ion battery salts, as well as new polymeric electrolytes that are amenable to mass production.

### BRIEF DESCRIPTION OF THE DRAWINGS

FIG. 1 is a plot showing plating and stripping of lithium on a Cu substrate and passivation of aluminum in a 1.0 molar LiP(C₂O₄)F₄ 1:1:3 PC-EC-EMC electrolyte, both of which are conducted at a scanning rate of 5 mV/s.

FIG. 2 is a plot showing voltage curves of the first cycle of Li-ion cells using an electrolyte of 1.0 molar LiP(C₂O₄)F₄ (solid line) and LiPF₆ (solid line interspersed with diamonds) dissolved in a 1:1:3 PC-EC-EMC mixture, both of which are recorded at 0.1 mA/cm².

### SUMMARY OF THE INVENTION

A compound active as an electrolyte has the formula

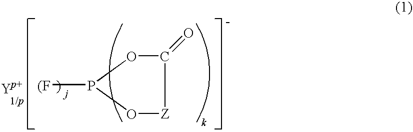

where p is an integer from 1 to 3 inclusive; and Y^{p+} is a metal ion, onium species, or proton; j is an integer value between 0 and 4 inclusive; k is an integer between 1 and 3 inclusive; and the sum 2k plus j equals 6; Z is independently in each occurrence CR¹R² or C(O); R¹ and R² are independently in each occurrence H, F or CH₃.

An oxyfluorophosphate compound is provided of the formula:

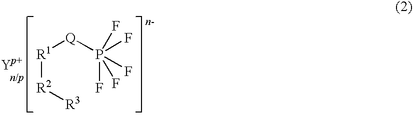

where Q is O, S or NR⁹; R¹ is C(O) or CR⁴R⁵; R² is

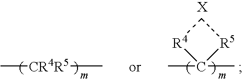

R³ is hydrogen, OM¹, SM¹, N(R⁹)₂, C(O)R⁶, CR⁴R⁵R⁶, or

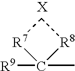

where R⁴ and R⁵ are independently in each occurrence hydrogen, halogen, C₁-C₈ alkyl, or C₀-C₈ alkyl halogen; R⁶ is hydrogen, OM¹, SM¹, or N(R⁹)₂; where R⁷ and R⁸ are independently in each occurrence hydrogen, halogen, fluorine or C₁-C₉ alkyl; where M¹ is hydrogen, a 1+ valency metal ion, or a quaternary ammonium cation; where R⁹ is independently in each occurrence hydrogen, fluorine, or C₁-C₈ alkyl; m is an integer from 0 to 6 inclusive; n is an integer from 1 to 3 inclusive; p is an integer from 1 to 3 inclusive; where X is a nullity, C₁-C₈ alkyl, or C₁-C₈ fluoroalkyl with the proviso that X is a nullity unless the pairs of R⁴ and R⁵, or R⁷ and R⁸ are both carbon containing and together with X form a five or six member ring; and Y^{p+} is a metal ion, onium species, or proton.

An operative electrolyte includes a polymeric phosphate salt electrolyte that includes a polymeric phosphate salt having a repeat unit formula:

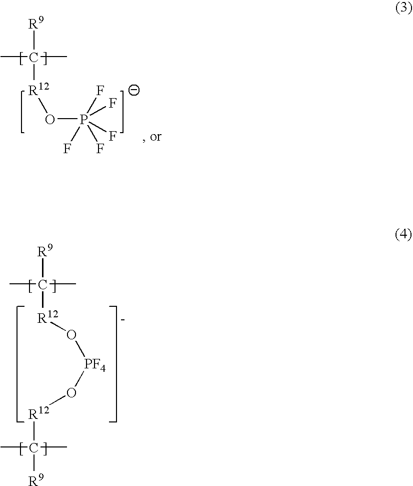

where R¹² is independently in each occurrence C(O), CR¹³R¹⁴C(O), or (CR¹³R¹⁴)_{q}; where R¹³ and R¹⁴ are independently in each occurrence hydrogen, C₁-C₈ alkyl, or C₀-C₈ haloalkyl, where R⁹ independently in each occurrence is hydrogen, fluorine, or C₁-C₈ alkyl; and where q is an integer between 0 and 2 inclusive; and an aprotic organic solvent or a polymer.

A process for preparing an oxyfluorophosphate includes the reaction of a phosphorus pentafluoro complex with a metal alkoxy, a polymer subunit, or an ester. Subsequent action of an oxyfluorophosphate produced by the reaction of a phosphorus pentafluoro complex with an ester through the exposure to a halide results in a salt well-suited as electrochemical device electrolyte salt.

The metal alkoxy operative herein has the formula: M²-O—R¹R²R³ where M² is an alkali metal, or alkali earth, R¹ is C(O) or CR⁴R⁵; R² is

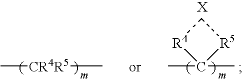

R³ is hydrogen, OM¹, SM¹, N(R⁹)₂, C(O)R⁶, CR⁴R⁵R⁶, or

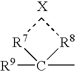

where R⁴ and R⁵ are independently in each occurrence hydrogen, halogen, C₁-C₈ alkyl, or C₀-C₈ alkyl halogen; R⁶ is hydrogen, OM¹, SM¹, or N(R⁹)₂; where R⁷ and R⁸ are independently in each occurrence hydrogen, halogen, fluorine or C₁-C₉ alkyl; where M¹ is hydrogen, a 1+ valency metal ion, or a quaternary ammonium cation; where R⁹ is independently in each occurrence hydrogen, fluorine, or C₁-C₈ alkyl; where X is a nullity, C₁-C₈ alkyl, or C₁-C₈ fluoroalkyl with the proviso that X is a nullity unless the pairs of R⁴ and R⁵, or R⁷ and R⁸ are both carbon containing and together with X form a five or six member ring; m is an integer from 0 to 6 inclusive. Alternatively, the oxygen of M²-O—R¹R²R³ is replaced with sulfur or NR⁹.

A further class of species reactive with phosphorus pentafluoride includes those pendent from a polymer chain. Such a subunit is either homo- or co-polymer that includes one or more of the following units:

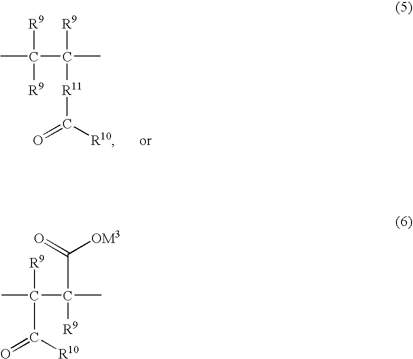

where R⁹ is independently in each occurrence a hydrogen, fluorine, or C₁-C₈ alkyl; and R¹⁰ is a hydrogen, fluorine, or OM⁴, and C₁-C₈ alkyl; R¹¹ is C₀-C₄ alkyl or C₆-C₁₀ aryl; M⁴ is hydrogen, a 1+ valency metal ion, C₁-C₄ alkyl, or a quaternary ammonium cation; and M³ is a 1+ valency metal ion.

An ester operative herein includes virtually any ester not precluded from reaction with a phosphorus pentafluoride complex by steric effects. Generally, the alkyl ester group according to the present invention is a C₁-C₈ alkyl group.

An inventive electrolyte is operative to produce an electrochemical device.

### DESCRIPTION OF THE PREFERRED EMBODIMENTS

The present invention has utility in the production of lithium ion electrolytes. According to the present invention, molecular and polymeric oxyfluorophosphates are formed through the process of reacting a phosphorus pentafluoride complex with a metal alkoxy or an ester. Through a robust and simple synthetic process, electrochemically stable lithium salts in particular are produced that have solubility in a variety of aprotic solvents conventional to the battery industry and provide high ionic conductivity over a variety of temperatures. As a result, inventive oxyfluorophosphates are operative to extend and/or improve the life and/or performance of electrochemical devices such as battery cells, capacitors, electrolytic cells, and supercapacitors.

A metal alkoxy operative according the present invention to react with a phosphorus pentafluoride complex has the formula: M²-O—R¹R²R³ where M² is an alkali metal, or alkali earth, R¹ is C(O) or CR⁴R⁵; R² is

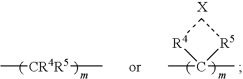

R³ is hydrogen, OM¹, SM¹, N(R⁹)₂, C(O)R⁶, CR⁴R⁵R⁶, or

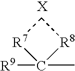

where R⁴ and R⁵ are independently in each occurrence hydrogen, halogen, C₁-C₈ alkyl, or C₀-C₈ alkyl halogen; R⁶ is hydrogen, OM¹, SM¹, or N(R⁹)₂; where R⁷ and R⁸ are independently in each occurrence hydrogen, halogen, fluorine or C₁-C₉ alkyl; where M¹ is hydrogen, a 1+ valency metal ion, or a quaternary ammonium cation; where R⁹ is independently in each occurrence hydrogen, fluorine, or C₁-C₈ alkyl; where X is a nullity, C₁-C₈ alkyl, or C₁-C₈ fluoroalkyl with the proviso that X is a nullity unless the pairs of R⁴ and R⁵, or R⁷ and R⁸ are both carbon containing and together with X form a five or six member ring; m is an integer from 0 to 6 inclusive. Alternatively, the oxygen of M²-O—R¹R²R³ is replaced with sulfur or NR⁹.

An oxyfluorophosphate compound is produced having the formula:

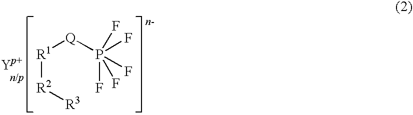

where Q is O, S or NR⁹; R¹ is C(O) or CR⁴R⁵; R² is

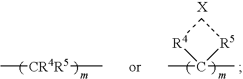

R³ is hydrogen, OM¹, SM¹, N(R⁹)₂, C(O)R⁶, CR⁴R⁵R⁶, or

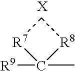

where R⁴ and R⁵ are independently in each occurrence hydrogen, halogen, C₁-C₈ alkyl, or C₀-C₈ alkyl halogen; R⁶ is hydrogen, OM¹, SM¹, or N(R⁹)₂; where R⁷ and R⁸ are independently in each occurrence hydrogen, halogen, fluorine or C₁-C₉ alkyl; where M¹ is hydrogen, a 1+ valency metal ion, or a quaternary ammonium cation; where R⁹ is independently in each occurrence hydrogen, fluorine, or C₁-C₈ alkyl; m is an integer from 0 to 6 inclusive; n is an integer from 1 to 3 inclusive; p is an integer from 1 to 3 inclusive; where X is a nullity, C₁-C₈ alkyl, or C₁-C₈ fluoroalkyl with the proviso that X is a nullity unless the pairs of R⁴ and R⁵, or R⁷ and R⁸ are both carbon containing and together with X form a five or six member ring; and Y^{p+} is a metal ion, onium species, or proton.

Specific examples of a metal alkoxy operative herein illustratively include bilithium oxalate, LiHC₂O₄, lithium alkyl oxalate, alkali metal salts of polyacrylic acid, including the sodium salt and lithium salt thereof, and a group of lithium salts, such as R¹R²C(OH)COOLi and R⁴R⁵C(OR⁴)COOLi.

The following polymer subunits react with PF₅ to form stable phosphorus-based polymeric salts and are readily converted to make single-ion (alkali metal ion, Li⁺) conducting polymer electrolyte by the technologies known to these who work in the art, illustratively including: blending with polymers such as poly(ethylene oxide); and plasticizing with aprotic solvents, as defined herein. Inventive polymers are either homo- or co-polymers include one or more of the following units:

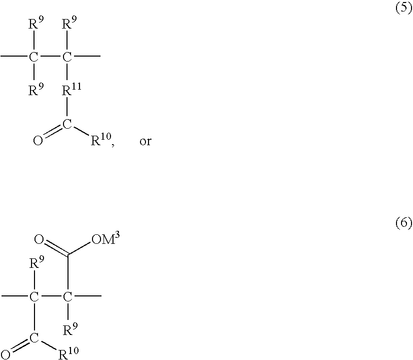

where R⁹ is independently in each occurrence a hydrogen, fluorine, or C₁-C₈ alkyl; and R¹⁰ is a hydrogen, fluorine, or OM⁴, and C₁-C₈ alkyl; R¹¹ is C₀-C₄ alkyl or C₆-C₁₀ aryl; M⁴ is hydrogen, a 1+ valency metal ion, C₁-C₄ alkyl, or a quaternary ammonium cation; and M³ is a 1+ valency metal ion. Preferably, M³ is lithium.

When R¹⁰ s OM⁴ and M⁴ is C₁-C₄ alkyl, the unit (5) or (6) is an ester. Specific examples of polymeric esters encompassing units (5) or (6) illustratively include ethoxylated polyacrylic acid, poly(alkyl acrylate), and poly(maleic acid ester).

Upon reaction with PF_{S}, polymer units (5) or (6) may form stable intermediates, which themselves serve as polymeric salts having the repeat unit formulas (3) or (4), respectively.

The reaction of a phosphorus pentafluoride complex with an ester according to the present invention, while not limited to a particular theory, is believed to occur through a metastasis reaction resulting in a linkage being formed between a carboxyl oxygen and phosphor with the ester alkyl group combining with fluoride to form a reaction byproduct.

Virtually any ester not precluded from reaction with a phosphorus pentafluoride complex by steric effects is operative herein. Generally, the alkyl ester group according to the present invention is a C₁-C₈ alkyl group. It is appreciated that a diester of a polycarboxylic acid is capable of forming cyclic or polymeric oxyfluorophosphates. In particular, the diester of oxalic acid forms a stable five-member ring structure having the formula C₂O₄ PF₄. Additionally, a mixed ester metal alkoxy dicarboxylate is appreciated to also be operative herein to form inventive compounds. Unlike the above-described reactions of metal alkoxy and phosphorus pentafluoride complex as according to the present invention, a reaction of an ester with a phosphorus pentafluoride complex

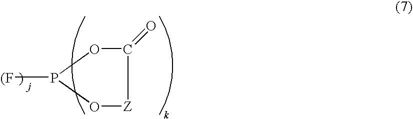

typically yields a net neutral charge reaction product of the formula:
where j is an integer value between 0 and 4 inclusive; k is an integer between 1 and 3 inclusive; and the sum 2k plus j equals 5; Z is independently in each occurrence CR¹R² or C(O); R¹ and R² are independently in each occurrence H, F or CH₃. The resulting product has utility as a chelating ligand.

A neutral product is rendered operative as an electrochemical device electrolyte through a subsequent reaction with a metal halide so as to form a salt of the formula (I).

It is appreciated that a variety of metal halides are operative herein to react with the neutral product. Metal halides operative herein illustratively include fluorides, chlorides, and bromides of: lithium; sodium; potassium; cesium; magnesium; calcium; strontium; transition metals such as silver, zinc, copper, cobalt, iron, nickel, manganese, titanium; metals from groups 13, 14 and 15 such as aluminum, gallium, tin, lead, and bismuth. Additionally, it is appreciated that an inventive neutral compound is also reacted with an organohalide illustratively including the fluoride, chloride or bromide salts of tetra-alkyl ammonium such as tetramethyl, tetraethyl, tetrabutyl, and triethylmethyl; pyridinium; imidazolium; tetra-alkyl phosphonium; tetra-aryl phosphonium; triaryl sulfonium, and trialkyl sulfonium. Preferably, the metal halide is lithium fluoride when the resulting compound is to be used as a lithium ion electrolyte.

A typical process for producing inventive oxyfluorophosphate according to the present invention includes combining the phosphorus pentafluoride complex and the metal alkoxy and/or ester in the absence of water at a temperature sufficient to allow volatilization of the heteroatom containing species of the complex. Typical reaction temperatures range from 0-200° C. The resulting product is dried to form a product corresponding to formula (I). Product drying occurs through heating to temperatures typically ranging from 20-200° C., optionally, while under vacuum. Subsequent purification is performed by techniques conventional to the art illustratively including solvent extraction and recrystallization.

Inventive oxyfluorophosphate is typically formed by the reaction of a phosphorus pentafluoride complex with either a metal alkoxy or an ester through interaction at one atmosphere in a solvent such as acetonitriles, ethers, tetrahydrofurans, carbonates, and mixtures thereof. Reaction occurs at temperatures generally ranging from 20° C. to the reflux temperature of the particular solvent. The resulting salt is isolated by conventional purification techniques. It is appreciated that reaction at different pressures is also operative with account for the pressure dependencies of solvent properties.

An inventive halogenated electrolyte is operative either in pure form or in combination with other salts known to those skilled in the art. An inventive halogenated phosphate is operative as an electrolyte salt in primary and secondary batteries, capacitors, super capacitors and electrolytic cells. The concentration of a halogenated phosphate according to the present invention in an electrolyte is typically between 0.01 and 3 molar, preferably from 0.01 to 2 molar, and most preferably from 0.1 to 1.5 molar.

An inventive halogenated phosphate is solvated to create an operative electrolyte. The solvent is a single, or preferably a mixture of aprotic solvents where aprotic solvents operative herein illustratively include (C₁-C₆ alkyl)—OC(O)—O—(C₁-C₆ alkyl), a C₂-C₈ alkaline carbonate, a C₁-C₆ dialkoxy of a C₂-C₆ alkane, a C₁-C₆ ester of a C₂-Cₛ carboxylic acid, a C₁-C₆ dialkyl sulfoxide, a C₀-C₆ alkyl tetrahydrofuran, a lactone, a pyrrolidinone, a nitrile, and mixtures thereof. Specific examples of aprotic solvents include dimethyl carbonate, diethyl carbonate, ethyl methyl carbonate, methyl propyl carbonate, propylene carbonate, ethylene carbonate, 1,2-dimethoxyethane, 1,2-diethoxyethane, methyl acetate, gamma-butyrolactone, ethyl acetate, methyl propionate, ethyl propionate, methyl butyrate, ethyl butyrate, dimethyl sulfoxide, dioxolane, sulfolane, 1-methyl-2-pyrrolidinone, acetonitrile, acrylonitrile, tetrahydrofuran, 2-methyltetrahydrofuran and mixtures thereof. According to the present invention, the electrolyte solvent is a mixture of at least one and preferably three solvents that function synergistically to solubilize inventive halogenated phosphate, promote thermal stability, and enhance ionic conductivity. Preferably, at least one of the solvents is an alkaline carbonate and a second solvent is (C₁-C₆ alkyl)—OC(O)—O—(C₁-C₆ alkyl). More preferably, the ratio of alkaline carbonate:(C₁-C₆ alkyl)—OC(O)—O—(C₁-C₆ alkyl) is in a ratio of from 0.1 to 10:1. Still more preferably, a lactone is present in the solvent mixture.

It is appreciated that the reaction of a phosphorus pentafluoride complex can occur neat or in the presence of a solvent. Suitable reaction solvents include aprotic organic solvents as detailed herein.

Through the reaction of a polymeric metal alkoxy and/or ester, phosphor fluoride containing polymeric salts are obtained that are particularly useful in creating alkaline metal ion conducting polymer electrolytes. The formation of such an electrolyte is well known to one skilled in the art and illustratively includes blending a polymeric salt with poly(ethylene oxide) and plasticizing the polymeric salt with an aprotic solvent as defined herein, and subsequently forming an interpenetrating polymer network through a secondary polymerization reaction around the polymeric salt.

The following non-limiting examples are provided to further illustrate the present invention. The examples are not intended to limit the scope of the present invention, but rather to detail specific aspects thereof.

### Example 1

### Synthesis of lithium oxalylfluorophosphates (LiOFP) including lithium oxalyltetrafluorophosphate (LiOTFP), lithium dioxalyldifluorophosphate (LiDODFP) and lithium trioxalylphosphate (LiTOP)

With exclusion of moisture, a Schlenk flask containing 45.6 g (0.3 mol) of LiPF₆ was heated at 180-200° C. to quantitatively produce PF₅ gas. The gas was transferred from the generator flask through a tube line to a reaction flask containing 30.6 g (0.3 mol) of lithium oxalate (Li₂C₂O₄) and 250 mL of acetonitrile and allowed to react with stirring for two hours. The resulting mixture was filtered and evaporated under a reduced pressure and further dried at 80-90° C. under vacuum for six hours to obtain a crude product. The crude product was purified by recrystallization using a 1:1 by volume ratio of acetonitrile to dimethyl carbonate. After drying for 16 hours under vacuum at 90° C., LiOTFP with high purity was obtained and identified by NMR spectroscopy as being LiP(C₂O₄)F₄. By changing the ratio of LiPF₆ to Li₂C₂O₄, LiP(C₂O₄)₂F₂ (LiPF₆/Li₂C₂O₄=1:2 in mol) and LiP(C₂O₄)₃ (LiPF₆/Li₂C₂O₄=1:3 in mol) also can be readily synthesized.

### Example 2

### Synthesis of lithium glycoltetrafluorophosphate (LiGTFP), lithium diglycoldifluorophosphate (LiDGDFP) and lithium triglycol phosphate (LiTGP)

With exclusion of moisture, a Schlenk flask containing 45.6 g (0.3 mol) of LiPF₆ was heated at 180-200° C. to quantitatively produce PF₅ gas. The gas was transferred from the generator flask through a tube line to a reaction flask containing 28.8 g (0.3 mol) of lithium methoxyacetate (CH₃OCH₂CO₂Li) and 250 mL of acetonitrile and allowed to react with stirring until getting a clear solution or for two hours. The resulting mixture was filtered and evaporated under a reduced pressure and further dried at 80-90° C. under vacuum for six hours to obtain a crude product. The crude product was purified by recrystallization using a 1:1 by volume of acetonitrile/dimethyl carbonate solvent. After drying for 16 hours under vacuum at 90° C., LiGTFP with high purity was obtained and identified by NMR spectroscopy as being LiP(OCH₂CO₂)F₄ (LiGTFP). By changing the ratio of LiPF₆ to CH₃OCH₂CO₂Li, LiP(OCH₂CO₂)₂F₂ (LiPF₆/CH₃OCH₂CO₂Li=1:2 in mol) and LiP(OCH₂CO₂)₃ (LiPF₆/CH₃OCH₂CO₂Li=1:3 in mol) also can be readily synthesized.

### Example 3

### Synthesis of LiOTFP or LiDODFP or LiTOP in the Absence of a Solvent

With exclusion of moisture, 45.6 g (0.3 mol) of LiPF₆ and 30.6 g (0.3 mol) of lithium oxalate (Li₂C₂O₄) were ground together, then sealed in a pressure vessel and heated at 150-180° C. for 1 hour. The resulting mixture was reground and extracted with 250 mL of acetonitrile. The resulting solution was evaporated under a reduced pressure and further dried at 80-100° C. under vacuum for six hours to obtain a crude product. The crude product was purified by recrystallization using a 1:1 by volume of acetonitrile/dimethyl carbonate solvent. After drying for 16 hours under vacuum at 90° C., LiOTFP with high purity was obtained and identified by NMR spectroscopy as being LiP(C₂O₄)F_{4 (LiOTFP). By changing the ratio of LiPF}₆ to Li₂C₂O₄, LiP(C₂O₄)₂F₂ (LiPF₆/Li₂C₂O₄-1:2 in mol) and LiP(C₂O₄)₃ (LiPF₆/Li₂C₂O₄=1:3 in mol) also can be readily synthesized.

### Example 4

### Synthesis of LiGTFP or LiDGDFP or LiTGP in the Absence of a Solvent

With exclusion of moisture, 45.6 g (0.3 mol) of LiPF₆ and 28.8 g (0.3 mol) of lithium methoxyacetate (CH₃OCH₂CO₂Li) were ground together, then sealed in a pressure vessel and heated at 150-180° C. for 1 hour. The resulting mixture was reground and extracted with 250 mL of acetonitrile. The resulting solution was evaporated under a reduced pressure and further dried at 80-100° C. under vacuum for six hours to obtain a crude product. The crude product was purified by recrystallization using a 1:1 by volume of acetonitrile/dimethyl carbonate solvent. After drying for 16 hours under vacuum at 90° C., LiGTFP with high purity was obtained and identified by NMR spectroscopy as being LiP(OCH₂CO₂)F₄ (LiGTFP). By changing the ratio of LiPF₆ to CH₃OCH₂CO₂Li, LiP(OCH₂CO₂)₂F₂ (LiPF₆/CH₃OCH₂CO₂Li=1:2 in mol) and LiP(OCH₂CO₂)_{3 (LiPF}₆/CH₃OCH₂CO₂Li=1:3 in mol) also can be readily synthesized.

### Example 5

### Alternate Synthesis of LiOTFP or LiDODFP or LiTOP in the Presence of a Solvent

With exclusion of moisture, 30.6 g (0.3 mol) of lithium oxalate (Li₂C₂O₄) was added to a Schlenk flask containing a solution consisting of 45.6 g (0.3 mol) of LiPF₆ and 250 mL of acetonitrile, and refluxed with stirring for two hours. The resulting mixture was filtered and evaporated under a reduced pressure and further dried at 80-90° C. under vacuum for six hours to obtain a crude product. The crude product was purified by recrystallization using a 1:1 by volume of acetonitrile/dimethyl carbonate solvent. After drying for 16 hours under vacuum at 90° C., LiOTFP with high purity was obtained and identified by NMR spectroscopy as being LiP(C₂O₄)F₄ (LiOTFP). By changing the ratio of LiPF₆ to Li₂C₂O₄, LiP(C₂O₄)₂F₂ (LiPF₆/Li₂C₂O₄-1:2 in mol) and LiP(C₂O₄)₃ (LiPF₆/Li₂C₂O₄-1:3 in mol) also can be readily synthesized.

### Example 6

### Alternate Synthesis of LiGTFP or LiDGDFP or LiTGP in the Presence of a Solvent

With exclusion of moisture, 28.8 g (0.3 mol) of lithium methoxyacetate (CH₃OCH₂CO₂Li) was added to a Schlenk flask containing a solution consisting of 45.6 g (0.3 mol) of LiPF₆ and 250 mL of acetonitrile, and flexed with a strong stirring for two hours. The resulting mixture was filtered and evaporated under a reduced pressure and further dried at 80-90° C. under vacuum for six hours to obtain a crude product. The crude product was purified by recrystallization using a 1:1 by volume of acetonitrile/dimethyl carbonate solvent. After drying for 16 hours under vacuum at 90° C., LiGTFP with high purity was obtained and identified by NMR spectroscopy as being LiP(OCH₂CO₂)F₄ (LiGTFP). By changing the ratio of LiPF₆ to CH₃OCH₂CO₂Li, LiP(OCH₂CO₂)₂F_{2 (LiPF}₆/CH₃OCH₂CO₂Li=1:2 in mol) and LiP(OCH₂CO₂)₃ (LiPF₆/CH₃OCH₂CO₂Li=1:3 in mol) also can be readily synthesized.

### Example 7

### LiOTFP Electrolyte and its Properties

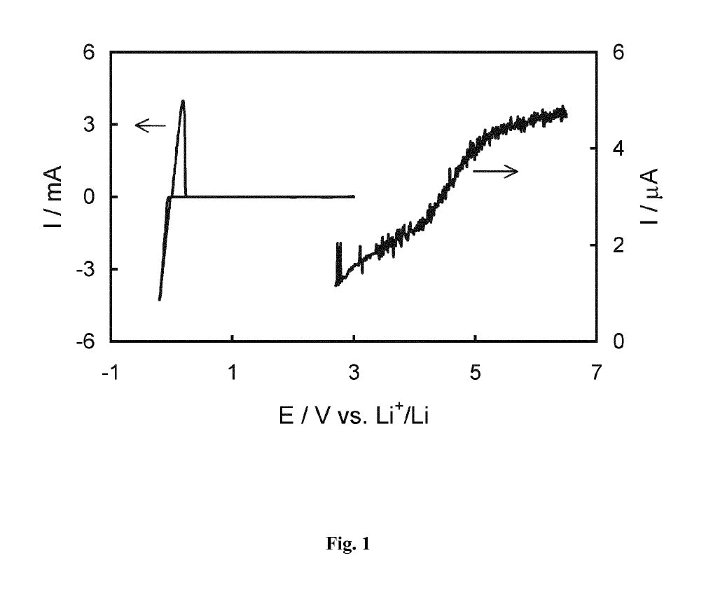

An electrolyte was prepared by dissolving 1.0 molar LiOTFP produced in example 1 into a 1:1:3 weight ratio mixture of propylene carbonate (PC), ethylene carbonate (EC), and ethyl methyl carbonate (EMC). Ionic conductivity of the electrolyte is determined to be 7.5 milliSiemens (mS)/cm at 22° C. by a means of measuring the impedance of a dip-type two-electrode cell. Ionic conductivity of the said electrolyte indicates that the LiOTFP is capable of providing high ionic conductivity. Cu and Al wires that both have a diameter of 0.1 cm and a length of 1.0 cm with a freshly scratched surface were exposed to the solution to determine cathodic and anodic stability of the electrolyte. Cu and Al were selected as being the most common materials for the current collector of the anode and cathode of lithium ion batteries. FIG. 1 shows cyclic voltammograms of the first scanning of a fresh Cu and Al, respectively, in a 1.0 molar LiOTFP 1:1:3 PC-EC-EMC electrolyte. It is shown that the plating and stripping of lithium metal reversibly takes place near 0 V vs. Li⁺/Li due to the presence of lithium ions in the solution, and that Al is well passivated at high potentials. The above results prove that the LiOTFP electrolyte is electrochemically stable for the operations of lithium ion batteries.

### Example 8

### Cycling Performance of the First Cycle of Li-Ion Cells

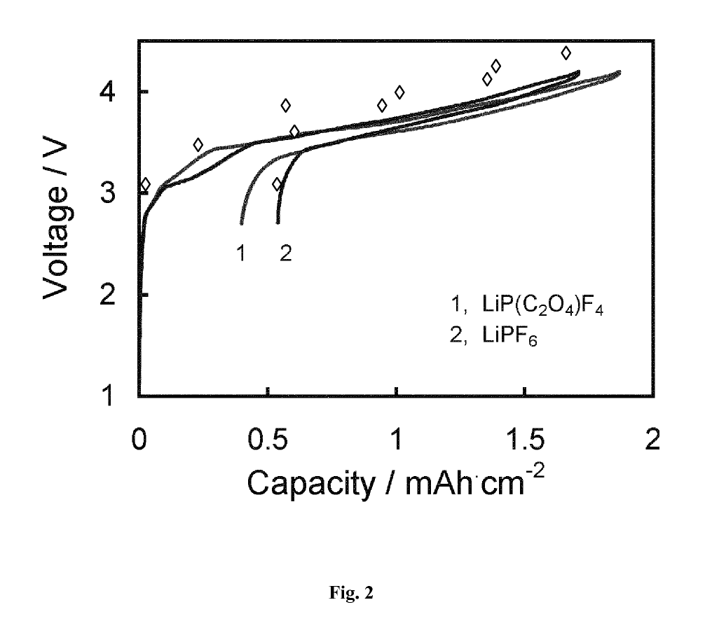

Two identical Li-ion cells using natural graphite anode and LiNi_{0.8}CO_{0.2}O₂ cathode were assembled. One cell was activated with an electrolyte as described in Example 7 and the other cell was activated with the same electrolyte but using LiPF₆ salt. Both cells were cycled at 0.1 mA/cm² by charging to 4.2 V and then discharging back to 2.5 V. FIG. 2 shows voltage curves of the first cycle of these two cells. It is determined that the cell using LiOTFP has a Coulomb efficiency of 79% and the one using LiPF₆ has only 69%. This result indicates that LiOTFP is superior to LiPF₆ in Li-ion batteries.

Any patents or publications mentioned in the specification are indicative of the levels of those skilled in the art to which the invention pertains. These patents and publications are herein incorporated by reference to the same extent as if each individual patent or publication was specifically and individually indicated to be incorporated by reference. The preceding figures and description illustrate the general principles of the present invention and some specific embodiments thereof. These are not intended to be a limitation upon the practice of the present invention since numerous modifications and variations will be readily apparent to one skilled in the art upon consideration of the drawings and description. The following claims, including all equivalents thereof, are intended to define the scope of the invention.

1. A process for preparing an oxyfluorophosphate comprising the step of:
reacting a phosphorus pentafluoride complex with a metal alkoxy or an ester to liberate the heteroatom containing species.

2. The process of claim 18 wherein said phosphorus pentafluoride complex is reacted with said metal alkoxy.

3. The process of claim 18 wherein said phosphorus pentafluoride complex is reacted with said ester.

4. The process of claim 19 wherein said metal alkoxy is a lithium oxalate.

5. The process of claim 18 wherein said ester compound is a diester of a dicarboxylic acid.

6. The process of claim 20 further comprising the step of exposing the oxyfluorophosphate to a halide under conditions suitable to create a halide salt thereof.

7. The process of claim 22 wherein said dicarboxylic acid is oxalic acid.

8. The process of claim 18 wherein said ester has a C₁-C₈ alkyl ester group.

9. The process of claim 19 wherein said metal alkoxy has the formula M²-O—R¹—R²—R³ where M² is an alkali metal, alkali earth, or quaternary ammonium cation; R¹ is C(O) or CR⁴R⁵; R² is 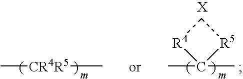
R³ is hydrogen, C(O)R⁶, CR⁴R⁵R⁶, or 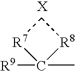
where R⁴ and R⁵ are independently in each occurrence hydrogen, halogen, C₁-C₈ alkyl, or C₀-C₈ alkyl halogen; R⁶ is hydrogen, OM¹, SM¹, or N(R⁹)₂; where R⁷ and R⁸ are independently in each occurrence hydrogen, halogen, fluorine or C₁-C₉ alkyl; where M′ is hydrogen, a 1+ valency metal ion, or a quaternary ammonium cation; where R⁹ is independently in each occurrence is hydrogen, fluorine, or C₁-C₈ alkyl; where X is a nullity, C₁-C₈ alkyl, or C₁-C₈ fluoroalkyl with the proviso that X is a nullity unless the pairs of R⁴ and R⁵, or R⁷ and R⁸ are both carbon containing and together with X form a five or six member ring; and m is an integer from 0 to 6 inclusive.

10. The process of claim 26 wherein R³ is C(O)R⁶ and R⁶ is OM¹.

11. The process of claim 26 wherein R³ is CR⁴R⁵R⁶, R⁴ is fluorine, R⁵ is fluorine and R⁶ is OM¹.

12. The process of claim 19 wherein said metal alkoxy has the repeating unit formula: 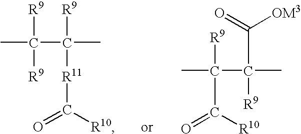
where R⁹ is independently in each occurrence a hydrogen, fluorine, or C₁-C₈ alkyl; R¹⁰ is a hydrogen, fluorine, OM⁴, or C₁-C₈ alkyl; R¹¹ is C₀-C₄ alkyl or C₆-C₁₀ aryl; M⁴ is hydrogen, a 1+ valency metal ion, C₁-C₄ alkyl, or a quaternary ammonium cation; and M³ is a 1+ valency metal ion.

13. The process of claim 29 wherein R⁹ is in each occurrence hydrogen.

14. The process of claim 29 wherein R¹⁰ is OM⁴ and M⁴ is lithium.

15. The process of claim 29 wherein M³ is lithium.

16. The process of claim 18 further comprising the step of recrystallizing said oxyphosphate.
